(19) United States
(12) Patent Application Publication
(10) Pub. No.: US 2017/0238806 A1
(43) Pub. Date: Aug. 24, 2017

(54) METHOD AND APPARATUS FOR IN-VIVO SPATIOTEMPORAL IMAGING OF TISSUE PROTEIN CLUSTERS

(71) Applicant: Toshiba America Electronic Components, Inc., San Jose, CA (US)

(72) Inventor: Rakesh SETHI, San Jose, CA (US)

(21) Appl. No.: 15/049,861

(22) Filed: Feb. 22, 2016

(51) Int. Cl. A61B 5/00; A61B 5/1459

(52) U.S. Cl. CPC A61B 5/0084; A61B 5/4058; A61B 5/4842; A61B 5/1459; A61B 5/0071

(57) ABSTRACT

A system and method for in-vivo monitoring for changes in body proteins over time includes an intelligent, implantable image capture system. An embedded processor controls activation of lighting for imaging proteins in surrounding tissue. A base image is captured and resulting data used in comparison with image data from one or more subsequent image captures to indicate progression of tissue changes, such as may occur with diseased tissue.

### TECHNICAL AREA

This application relates generally to a method and apparatus for clinical diagnosis. The application relates more specifically to an implantable sensor for detection and repair of progressive physiological conditions.

### BACKGROUND

Systems and methods of observing physical maladies continue to evolve rapidly. Earliest observations as to a patient's physical condition involved visual inspection by a clinician. Systems evolved to include analysis by surgical exploration and by testing physical properties of a subject or a subject's tissue. More recently, clinicians use devices such as X-ray, computed tomography (CT), magnetic resonance imaging (MRI), positron emission tomography (PET) and near-infrared spectroscopy (NIRS) for medical diagnostics.

Tools such as those noted above are useful to facilitate patient diagnostics and to provide a clinician with information about a patient's current physical condition.

### SUMMARY

In accordance with an example embodiment of the subject application, an implantable diagnostic system includes a power supply and an electromagnetic wave generator configured to generate an electromagnetic wave to biological tissue. An image sensor captures an image from the electromagnetic wave generator after exposure of the biological tissue. An analog front end circuit generates image data from a captured image. The electromagnetic wave generator is configured to be selectively enabled by the processor. The processor completes a plurality of image capture operations and stores image data in the memory for image captures. The processor then compares image data from an earlier image capture to a subsequent image capture and generates comparison data which is then output.

### BRIEF DESCRIPTION OF THE DRAWINGS

Various embodiments will become better understood with regard to the following description, appended claims and accompanying drawings wherein:

FIG. 1 is an example embodiment of a medical diagnostic or monitoring or repair system;

FIG. 2 is an example embodiment of a biosensor;

FIG. 3 is an example embodiment of an imaging system;

FIG. 4 is an example embodiment of a CMOS image capture system;

FIG. 5 is an example embodiment of a filter;

FIG. 6 is a flowchart of example operations of a biosensor; and

FIGS. 7-9 are charts that graphically illustrate example characteristics of biosensor imaging.

### DETAILED DESCRIPTION

The systems and methods disclosed herein are described in detail by way of examples and with reference to the figures. It will be appreciated that modifications to disclosed and described examples, arrangements, configurations, components, elements, apparatuses, devices methods, systems, etc. can suitably be made and may be desired for a specific application. In this disclosure, any identification of specific techniques, arrangements, etc. are either related to a specific example presented or are merely a general description of such a technique, arrangement, etc. Identifications of specific details or examples are not intended to be, and should not be, construed as mandatory or limiting unless specifically designated as such.

In accordance with example embodiments of the subject application, a system and method for improved analysis of human tissue facilitates detection and monitoring of a patient's current physical condition that may degrade or otherwise vary over time. Progress of a condition may be unique to a particular patient. Analysis may also target a particular condition by modifying aspects of the system to target it.

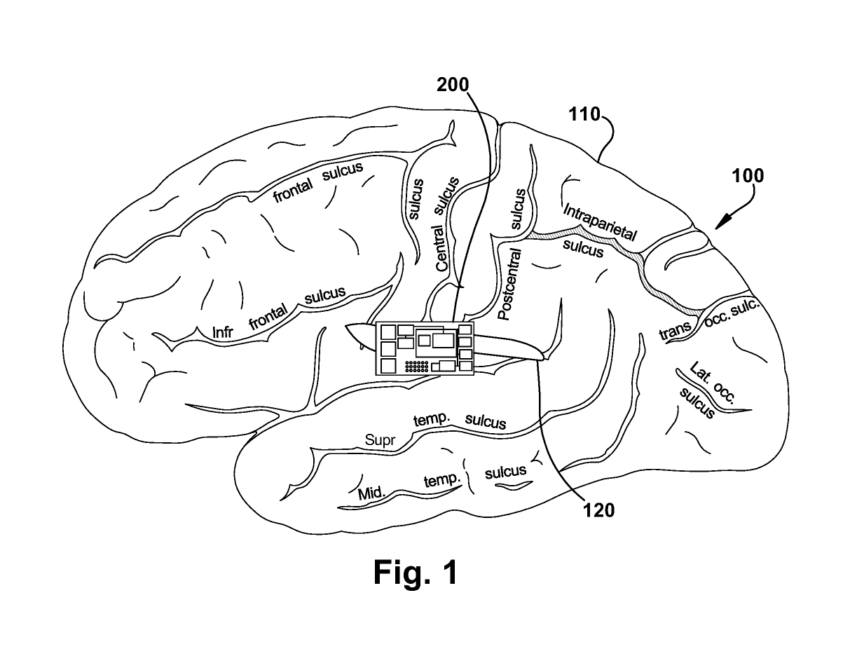

FIG. 1 illustrates an example embodiment of a medical diagnostic or monitoring system 100. In the illustrated example, various sulcus regions, depressions and grooves in the brain, are shown in a human brain 110. It will be appreciated that the brain is used for illustration of an example embodiment and that the biosensor 200 can suitably be applied to, implanted or embedded with any suitably tissue facilitating in-vivo monitoring of patients or subjects. In brain physiology, one or more protein islands 120 can signify an accumulation of proteins such as beta amolyd or Tau proteins. Such protein islands 120 may also contain a marker, such as oxytocin. In the illustrated example, biosensor 200 is placed on brain surface tissue, and may suitably be disposed so as to contact protein island 120. Oxytocin is detected in connection with the example embodiment herein, but it will be understood that any suitable marker can be used.

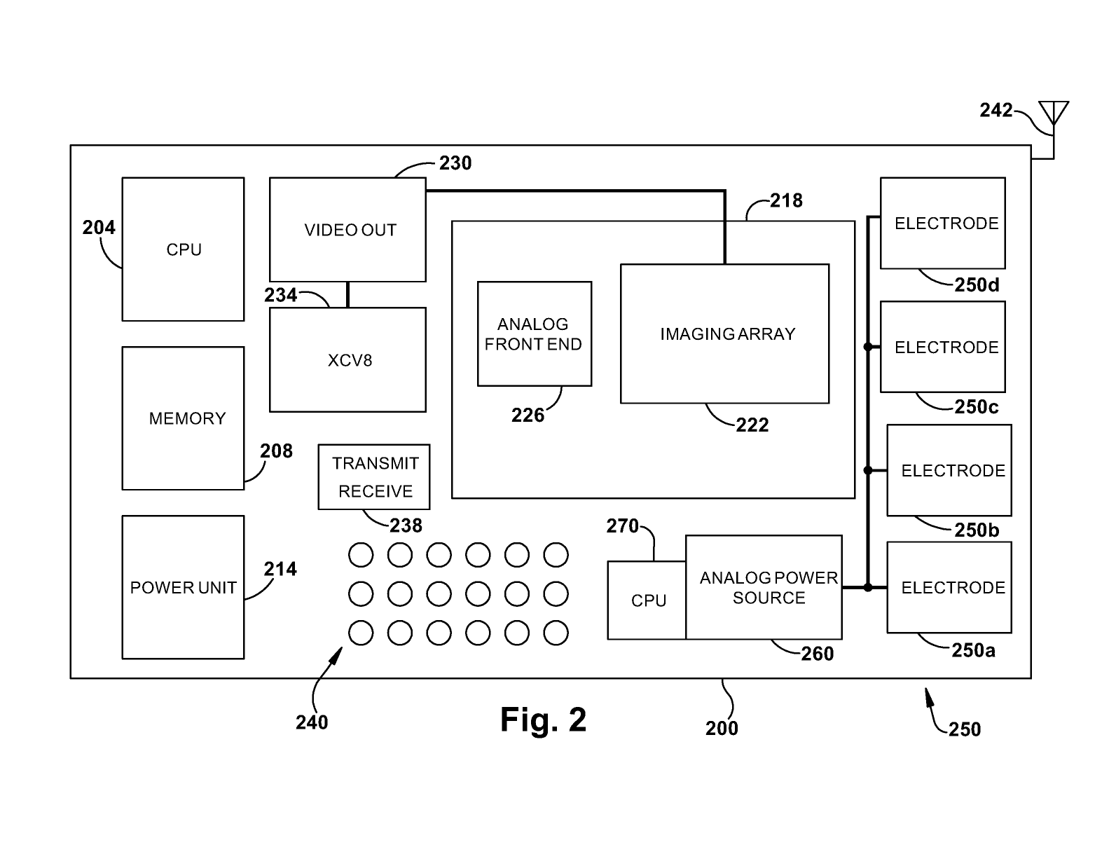

Turning now to FIG. 2, illustrated is an example embodiment of biosensor 200 that suitably includes CPU 204, memory 208 and power unit 214. Power unit 214 is suitably comprised of any suitable power source, such as a battery. It will be appreciated that power can also be supplied by a generator operable from ambient heat or via wireless power transmission. Biosensor 200 includes an imaging system 218, comprised of an imaging array 222 suitably formed from a solid state image capture array such as a charge-coupled device (CCD) array, suitably including a lensing system as will be understood by one of ordinary skill in the art. Imaging array 222 works in concert with an analog front-end (AFE) 226. AFE 226 is comprised of analog signal conditioning circuitry that suitably uses operational amplifiers and filters to provide a configurable and flexible processing module, and which functions to interface with sensors and perform analog to digital conversion. In the illustrated example, AFE 226 interfaces with imaging array 222 to perform digital image capture. By way of further example, AFEs include Texas Instruments products ADS 1298, AFE 440 and AFE 460.

By way of further example. AFE 226 suitably includes a detection circuit sensitive to flux intensity signals that are associated with monitoring or detection of specific tissue conditions as will be detailed further below. In a particular example embodiment, the AFE 226 includes a programmable amplifier having a set effective number of bits (ENOB) for analog to digital (A/D) conversion, such as a 20 bit resolution and high dynamic range above 100 dB. When working in concert with imaging array 222, for example as would be implemented in a CCD camera, the AFE 226 suitably shares a set of columns with the imaging array 222. Implementation of AFE 226 facilitates detection of low level fluorescence to facilitate monitoring and detection as illustrated further below.

Imaging system 218 suitably includes a filtering system for selecting one or more electromagnetic wave frequencies for attenuation or enhancement to enhance specific detection operations specific to a particular condition. Suitable imaging filtering systems will be detailed further below. Imaging system 218 interfaces with a video output 230 to facilitate image capture and processing, suitably controlled or tuned, with field programmable gate array 234 (FPGA), such as XCV8 series FPGAs. Tuning of image capture, suitably coupled with filtering of electromagnetic input, such as light, allows for capturing of diagnostic images for specific conditions as will be detailed further below. A transmitter or receiver unit 238 facilitates data communication, such as via a wireless data communication via antenna 242. In a configuration, wireless power transmission can be accomplished via application of oscillating magnetic waves to antenna 242.

The example embodiment of FIG. 2 facilitates capture of a series of images. An initial or earlier captured image has a corresponding value suitably stored in memory 208 as a baseline value. Image values from subsequently captured images are compared, and physiological changes detected as a result of such comparison. Detection of changes over time, particularly in connection with a malady to which the system is filtered and tuned, allows for diagnostics over time to detect maladies that may not otherwise be detected at all.

FIG. 2 also illustrates a light generation system suitably comprised of an electromagnetic wave illumination system, illustrated as light-emitting diode (LED) array 240 suitably powered by power unit 214. LED array 240 is enabled under control of one more processors, such as CPU 204, as is imaging system 218. Thus, control of lighting and image capture is choreographed by the CPU 204 so as to provide for capture of a series of images over a duration, such as a preset interval, while minimizing power drain.

Biosensor 200 suitably includes one or more electrodes 250, illustrated as electrodes 250a, 250b, 250c and 250d. Electrodes 250 are suitably powered by analog power source 260, which is suitably formed by inverting DC voltage which may be presented by power unit 214. Analog power source 260 is suitably under CPU control, such as CPU 204, or by a dedicated CPU controller 270. Electrodes 250 facilitate tissue stimulation for further diagnostics or treatment of tissue maladies. By way of further example, the electrodes 250 suitably provide open or closed loop control and delivery of electrical impulses. This may be done pre-imaging, during imaging, or post-imaging, depending on the particular application and target protein or proteins. As will be detailed further below, biosensor 200 suitably acts in conjunction with a filter, such as a light filter. Tuning for imaging one or more particular proteins is suitably accomplished by a combination of filtering and tuning. Filter characteristics input to the CPU 204 for processing, combined with protein characteristics, facilitate tuning the imaging array 222, such as by block, row, column or pixel level. Tuning suitably includes adjustment of illumination, such as via control of LED array 240. Available control options include selective illumination of individual or groups of LEDs. The LED array 240 is further suitably comprised of LED elements having different spectral outputs. Thus, selective enablement of elements or element intensity allows for engineered illumination to target specific proteins. As noted above, this is particularly advantageous when coupled with filter properties engineered for detection of one or more target proteins.

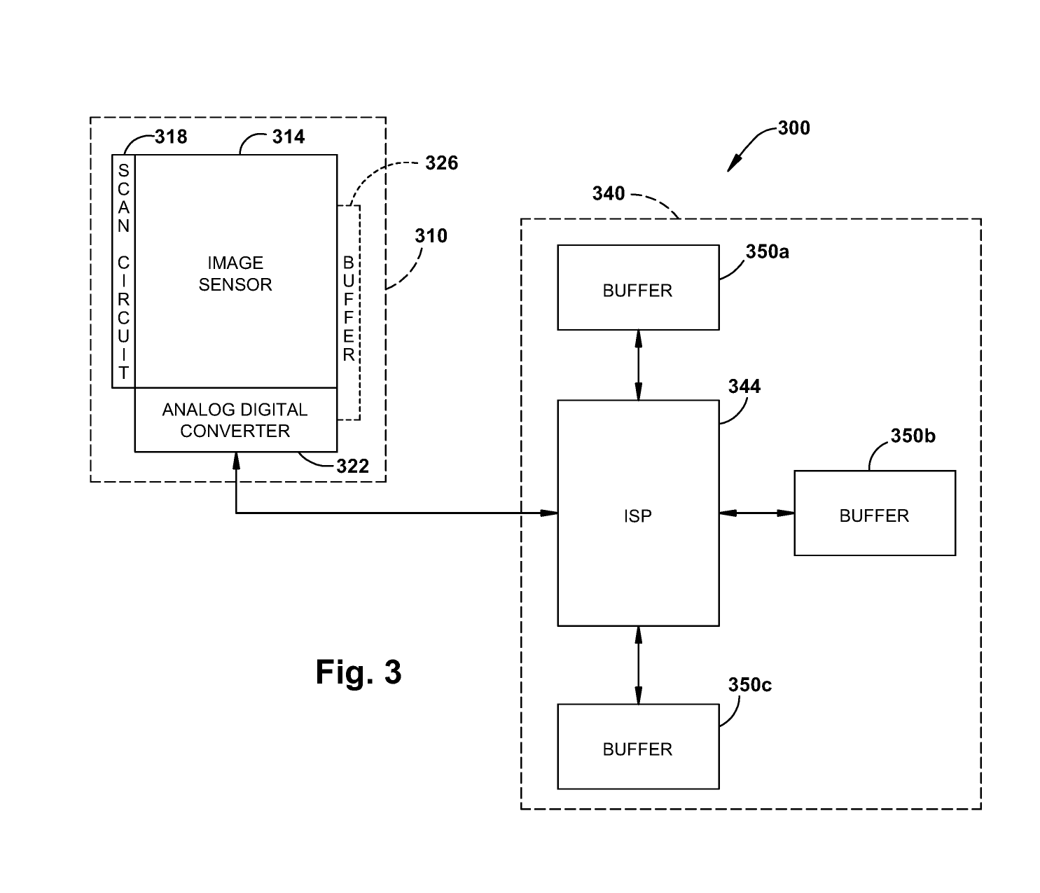

Referring now to FIG. 3, illustrated is an example embodiment of an imaging system 300. An image capture system 310 includes an image sensor 314, suitably coupled with scan circuit 318, A/D converter 322 and buffer 326. The image capture system 310 can include an array such as imaging array 222 of FIG. 2. Captured digital images are communicated to image processing system 340 for further processing by image signal processor 344, suitably in concert with one or more buffers such as buffers 350a, 350b and 350c.

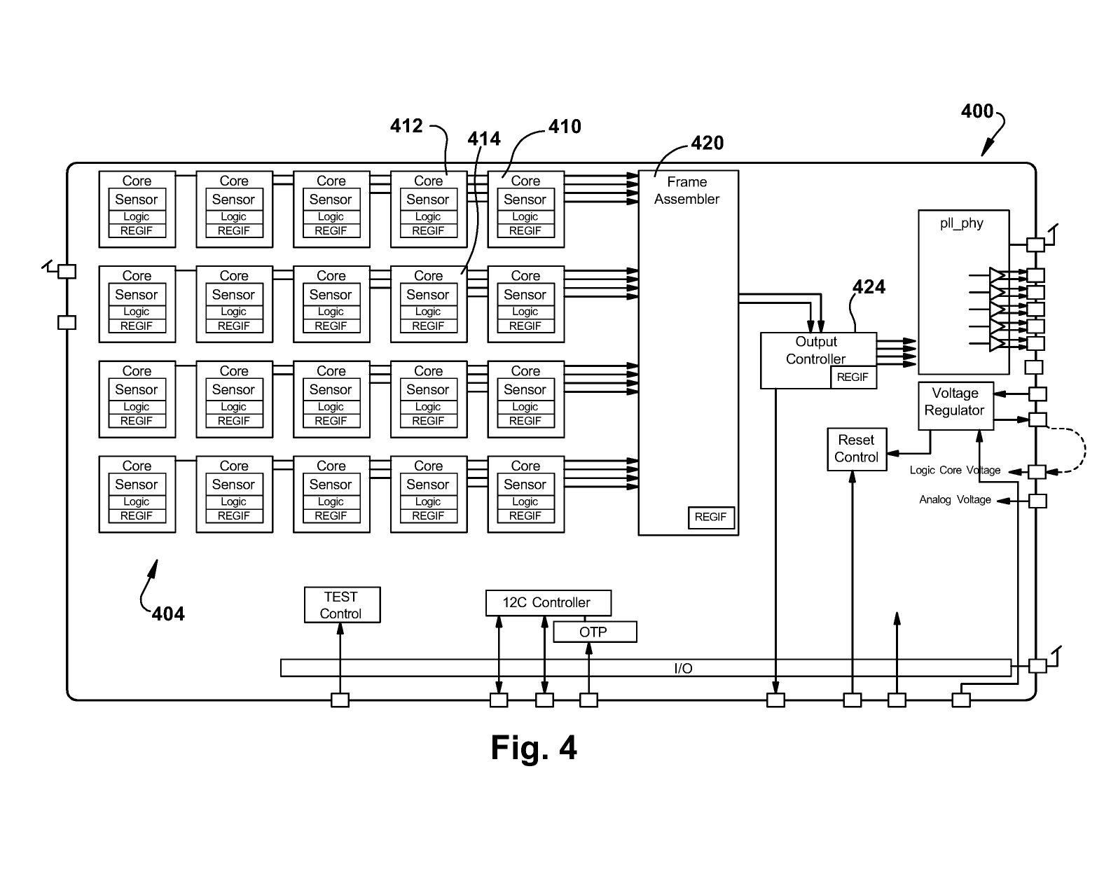

FIG. 4 illustrates an example embodiment of a CMOS image capture system 400. As will be appreciated by one of ordinary skill in the art, the example CMOS image capture system 400 includes CCD array 404, suitably comprised of sensor elements for complementary primary color image capture, such as capturing of red, green and blue spectral components. This is suitably accomplished by capturing pixels for the primary colors with neighboring CCD elements, such as red element 410, green element 412 and blue element 414. Captured images are suitably compiled within frame assembler 420 and communicated via output controller 424. Image capture system 400 suitably includes secondary support circuitry as illustrated and as will be understood by one of ordinary skill in the art.

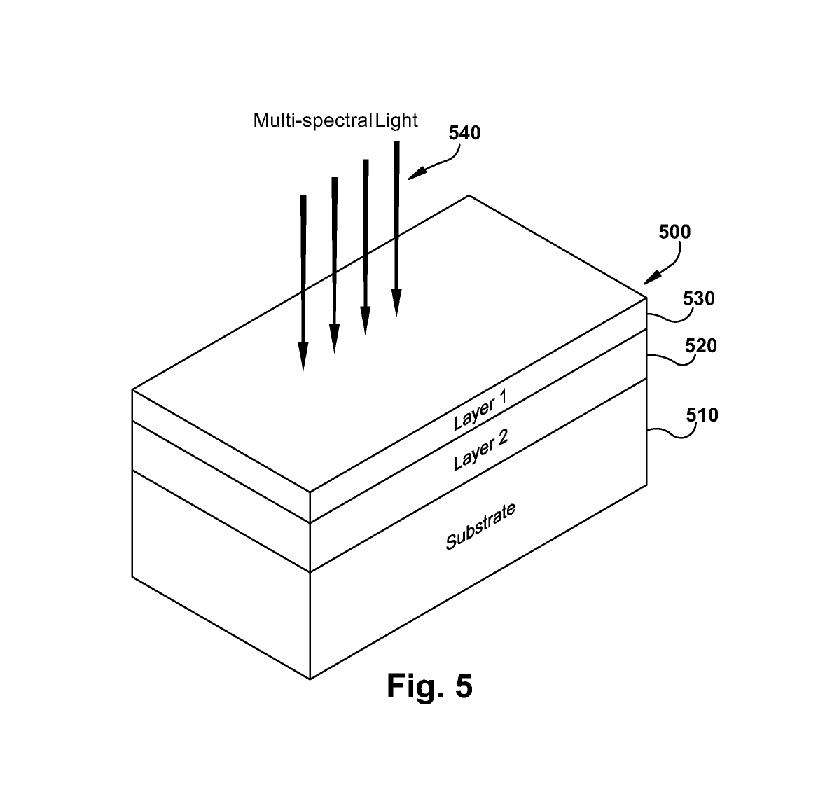

FIG. 5 illustrates an example embodiment of a filter 500, suitably disposed in conjunction with light communicated to an image capture array and lensing system, such as a CCD camera. Filtering via a light transmissive medium facilitates removal of one or more frequencies that may be unrelated to a particular condition and the presence of which would hinder capture of light of interest to a particular condition. Similarly, filter properties are suitably altered to accentuate frequencies that may be particularly associated with a particular condition. By way of particular example, oxytocin is associated with many physical conditions, including autism. Beta amolyd protein may be associated with dementia, Huntington disease or other disorders, and TAU protein may be associated with Alzheimer's disease. Oxytocin is associated with a yellow signal correlation centered around a wavelength of approximately 405 nm. Accordingly, detection of autism can be suitably accomplished by filtration and tuning for light in the yellow spectrum. Further refinement is suitably accomplished by addition of dyes or other substances that facilitate further refinement.

Analysis of captured images allows for medical diagnoses, but is also suitably used in connection with dosing or electrical stimulation for treatment. In connection with monitoring of brain activities, brains are associated with biological waste management processes that degrade over time, which degradation is detectable by the subject system. The subject system suitably includes closed-loop control to compensate for such degradation.

While filter 500 is suitably comprised of any electromagnetic wave transmissive medium, properties which allow one or more selected wavelengths, or wavelength ranges, are advantageously used in conjunction with imaging different protein characteristics. In the example embodiment of FIG. 5, filter 500 is comprise of a substrate 510 onto which is deposited one or more layers, illustrated as layer 520 and layer 530. In another embodiment, the filter is pseudomorphic for engineered filtering characteristics. Layer properties, as well as thicknesses, are suitably configured in accordance with a particular application for one or more target proteins. Engineered filters, such as pseudomorphic filters that use pseudomorphic crystals, are suitably comprised of one or more layers formed by spin-on-glass deposition on a substrate. The substrate may be comprised of a semiconductor, such as a CMOS wafer and may comprise deposition on a CCD array. Such deposition may also comprise lensing relative to camera pixel capture elements. In the example embodiment of FIG. 5, electromagnetic radiation, such as multi-spectra light 540 is reflected from or passed through a protein of interest. Layer 530 is engineered for light transmission and amplification, at least in a particular optical band, such as yellow. Light amplification may be accomplished by doping constituents, and energy for amplification in a certain band may come from energy in other bands. Layer 520 is engineered to filter light from wavelengths other than that desired. For example, layer 520 can filter infrared light. Thus, the light is communicated through translucent or transparent layer 510 before being communicated to a light sensor, such as a CCD pixel.

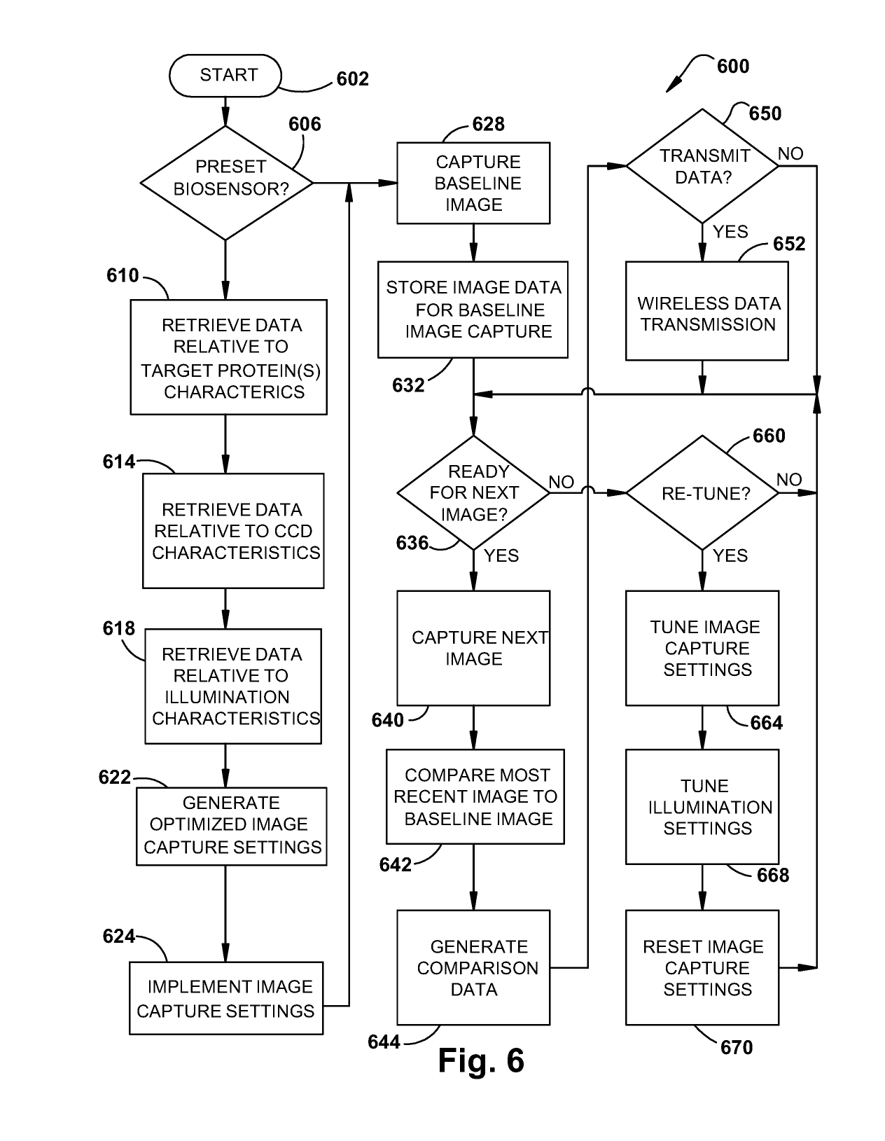

Turning now to FIG. 6, illustrated is an example embodiment of a flowchart 600 suitably implemented in code running on one or more processors of biosensor 200. The process suitably commences at block 602 and proceeds to block 606 where a determination is made as to whether the biosensor is ready for imaging or whether it is to be preset. For presetting, progression is made to block 610 and data is retrieved relative to characteristics of one or more proteins of interest. Next, CCD characteristic data is retrieved at block 614 and illumination characteristic data is obtained at block 618. Any or all of this data is used to generate optimized image capture settings at block 622, and these settings are implemented as image capture settings for the device at block 624. A baseline image capture is made at block 628. In the event that no presetting is to occur, progress is made directly from block 606 to block 628.

After a baseline image has been captured at block 628, the associated data is stored at block 632 and a determination is made at block 636 as to whether a new image should be captured. This may be determined, by way of example, by passing of a preselected duration from a prior image capture. If a determination is made that a new image should not yet be captured, a test for possible tuning or re-tuning is made at block 660. If so, image capture settings are tuned at block 664 and illumination settings are tuned at block 668. These new settings are implemented at block 670, and progress returns to block 636 to determine if it is time for the next imaging operation.

If, at block 636, a new image should be captured, then progress is made to block 640 and an image is captured. Progress is then made to block 642 where the captured image is compared to the baseline image. Progress is then made to block 644 where comparison data is generated based on the compare operation of block 642, and in block 650 a determination is made as to whether to transmit the data. If so, the data is wirelessly transmitted in block 652 and progress returns to block 636 to await the next imaging operation. If not, then progress returns to block 636 without wireless transmitting the data.

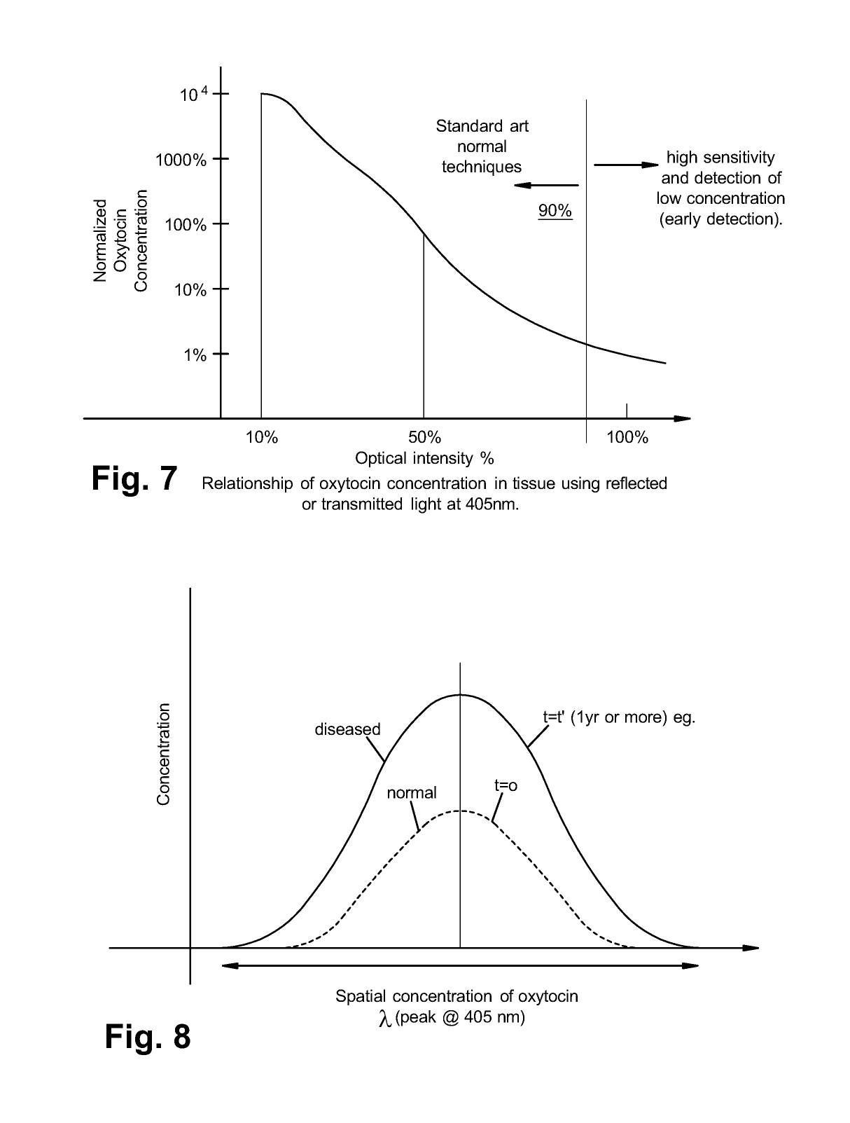

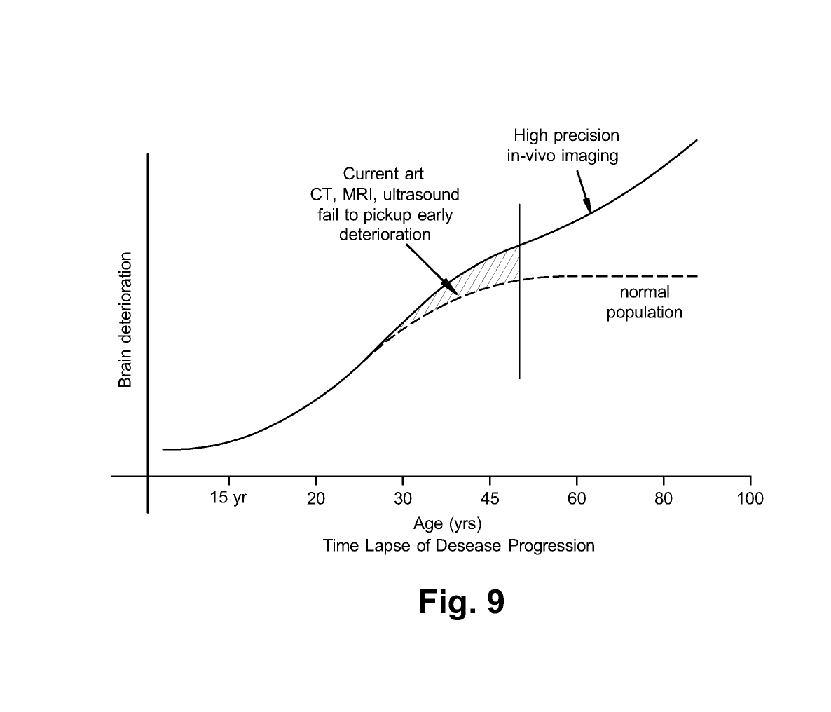

FIGS. 7-9 illustrate graphically example embodiments certain of the characteristics in connection with the description above. FIG. 7 shoes a relationship of oxytocin concentration with reflected or luminescent light from tissue. By way of example, light of interest may be in the 405 nm range. FIG. 8 shows a time lapse of disease progression as determined by a sequence of image captures as detailed above. FIG. 9 shows brain deterioration by measured values relative to established values of a normal population. The shaded area indicating early deterioration levels that current art CT, MRI, and ultrasound typically fail to detect. These relationships are suitably used to detect one or more maladies in a subject into which a biosensor has been implanted.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the spirit and scope of the inventions.

What is claimed is:

1. An implantable diagnostic and therapeutic system comprising:
a power supply;
an electromagnetic wave generator configured to generate an electromagnetic wave to neighboring biological tissue;
an image sensor configured to capture an image from the biological tissue after exposure to the electromagnetic wave from the generator;
an analog front end circuit configured to generate image data from a captured image;
a processor and associated memory,
the processor configured to selectively enable the electromagnetic wave generator,
the processor configured to initiate a plurality of image capture operations using the electromagnetic wave generator and the image sensor,
the processor configured to store image data in the memory for the image capture operations,
the processor configured compare image data from the image capture operations,
the processor configured to store comparison data generated from compared captured image data; and
a data output configured to output the comparison data.

2. The system of claim 1 wherein the image sensor includes a columnar CMOS sensor array, and
wherein the analog front end circuit is comprised of a programmable gain amplifier associated with a column of the sensor array.

3. The system of claim 1 wherein the electromagnetic wave generator is comprised of a light generator, and
wherein the image sensor includes a filter comprised of at least one layer configured to pass selected frequencies of light.

4. The system of claim 3 wherein the filter is comprised of a pseudomorphic crystal.

5. The system of claim 3 wherein the processor is further configured to tune the sensor array in accordance with a property of the filter.

6. The system of claim 3 wherein the filter is comprised of a spin-on-glass deposition configured to impede passage of infrared light.

7. The system of claim 1 wherein the biological tissue is comprised of protein, and
wherein the electromagnetic wave generator includes an electrode configured to generate a voltage relative to the biological tissue, and
wherein the processor configured to complete the plurality of image capture operations during operation in accordance with operation of the electrode.

8. A method comprising:
generating an electromagnetic wave in an implanted biosensor;
directing the electromagnetic wave to biological tissue adjacent the biosensor;
capturing, by an image sensor of the biosensor, an image from the biological tissue during exposure to the electromagnetic wave;
generating image data from a captured image via an analog front end of the biosensor;
completing a plurality of image capture operations using the electromagnetic wave generator and the image sensor;
storing image data in a memory of the biosensor for the image capture operations;
comparing image data from two or more image capture operations;
storing comparison data generated from compared image data; and
outputting the comparison data via a wireless data communication medium.

9. The method of claim 8 wherein generating the electromagnetic wave further comprises generating light, and further comprising:
filtering light through at least one layered substrate configured to pass selected frequencies of light to at least a portion of the image sensor.

10. The method of claim 9 wherein filtering light further comprises filtering light via a pseudomorphic crystal.

11. The method of claim 9 wherein filtering light further comprises filtering light via a spin-on-glass deposition configured to impede passage of infrared light.

12. The method of claim 9 wherein the image sensor includes a columnar CMOS sensor array, and further comprising:
processing the captured image via the analog front end circuit, and
wherein the analog front end circuit is comprised of a programmable gain amplifier associated with a column of the sensor array.

13. The method of claim 12 further comprising tuning the sensor array in accordance with a property of the filter.

14. The method of claim 8 wherein the biological tissue is comprised of proteins, and further comprising:
generating a voltage relative to the biological tissue;
applying the voltage to the biological tissue via an electrode associated with the biosensor; and
capturing a plurality of images during application of the voltage by the electrode.

15. An implantable biosensor system comprising:
a power supply;
a processor;
a memory;
a digital image capture array including an optical input;
a filter associated with the optical input and configured to pass one or more selected light frequencies;
an analog front end circuit configured to process captured images;
a light producing element;
a processor configured to selectively enable the light producing element and the digital image capture array to complete a plurality of image capture operations,
the processor configured to store, in the memory, image data corresponding to a primary image capture operation as baseline image data,
the processor configured to store, in the memory, image data corresponding to subsequent image capture operations, and
the processor configured to compare image data from subsequent image capture operations to baseline image data and generate comparison data; and
a wireless data interface configured to output comparison data.

16. The system of claim 15 wherein the filter is comprised of a spin-on-glass deposition substrate.

17. The system of claim 15 wherein the filter is comprised of a pseudomorphic crystal.

18. The system of claim 17 further comprising a tuner configured to tune the digital image capture array in accordance with a property of the pseudomorphic crystal.

19. The system of claim 15 wherein the analog front end is comprised of a programmable gain amplifier.

20. The system of claim 15 wherein the filter is configured to accentuate selected light frequencies.

21. The system of claim 1 further comprising the processor configured to modulate and monitor dynamic response of a vascular system via the wave generator.
